(19) United States
(12) Patent Application Publication
(10) Pub. No.: US 2011/0264095 A1
(43) Pub. Date: Oct. 27, 2011

(54) Assisted Positioning Device for Bone Plate

(75) Inventors: Cheng-Kung Cheng, Taipei (TW); Yu-Shu Lai, Taipei (TW); Yu-Liang Liu, Taipei (TW)

(73) Assignee: Cheng-Kung Cheng, Taipei (TW)

(21) Appl. No.: 12/853,143

(22) Filed: Aug. 9, 2010

(30) Foreign Application Priority Data
Apr. 26, 2010 (TW) 099113021

(51) Int. Cl. A61B 17/80

(52) U.S. Cl. 606/71

(57) ABSTRACT

The present invention relates to an assisted positioning device for bone plate, including: a bone plate, a first positioning component and a second positioning component. The surface of the bone plate configured a number of positioning grooves in order to correspond with a number of bone screws provided for the integration. One end portion of the bone plate configures a first positioning slot, and the first positioning slot is containing a gap. When the end portion of the first positioning component combined the first positioning slot, a first positioning pin can be penetrated by a first through slot of the first positioning component, and in line with the gap. The device can actually reduce the size of incision, shorten the healing time after surgery, and have precisely positioning capabilities.

### BACKGROUND OF THE INVENTION

1. Field of the Invention

The present invention relates to a device for bone plate. In particular, the present invention is an assisted positioning device for bone plate which can reduce the size of patient's incision and shorten healing time after surgery.

2. Prior Art

When having a traffic accident or falling down, human skins and bones are often hit by impact force directly. If it's minor, people would only have flesh wounds, but if it's serious, people would have a break in bones which is known as “bone fracture”. Besides wearing the traditional plasters cast or splints, the most common way to deal with the fracture is to use bone screw and bone plate to fix it through surgery. But the surgical equipment is too heavy, expensive, and complicated to operate. It not only wastes the medical sources but also raises the risk because of the long surgery time.

FIG. 1 is a side view illustrating a surgical method of bone fracture from U.S. Pat. No. 5,951,557. As shown in FIG. 1, when patient's arm 10 bone 11 fractured, paramedics need to approach an incision 12 in patient's arm 10 and use medical equipment 20 to expand the incision 12 for showing the fractured bone 11.And then inserting the bone plate 30 through the incision 12, using numbers of screw holes 31 to combine the fractured bone 11 and stitch the incision 12 to allow the fractured bone 11 to heal by itself. It is not very pragmatic for this kind of surgery because it needs to cut a larger incision 12 to position bone 11 which could be painful for a long time, and take a longer healing time after surgery.

Therefore, it's meaningful to think how to reduce the incision size during bone fracture surgery, shorten the healing time and have precisely positioning capabilities.

### SUMMARY OF THE INVENTION

One aspect of the present invention is to provide an assisted positioning device for bone plate which can reduce the size of incision, shorten healing time and have precisely positioning capabilities.

In order to achieve this aim, the present invention includes an assisted positioning device for bone plate comprising:
a bone plate, having several positioning grooves in the surface in order to correspond with a number of bone screws provided for the integration, and one end portion of the bone plate configures a first positioning slot, and the other end portion of the bone plate configures a second positioning slot, and the first positioning slot includes a gap;
a first positioning component, having a first through slot configured in central axis of the first positioning component, and the first through slot can allow a first positioning pin to insert inside. When one end portion of said first positioning component is combined with the first positioning slot, the first positioning pin can penetrate the first through slot and the first positioning slot. The first positioning pin size is smaller than the gap; a second positioning component, having a first end portion and a second end portion, and the second positioning component configures a second through slot in central axis of the second positioning component to connect the first end portion and the second end portion and the second through slot can allow a second positioning pin to insert inside. When the first end portion of the second positioning component is combined with the second positioning slot, the second positioning pin can penetrate the second through slot and the second positioning slot. The second end portion size is smaller than the second positioning slot.

According to one embodiment of the present invention, said first positioning slot further includes a central slot and at least one spacing hole, and said first positioning component further includes at least one positioning convex which can be inserted respectively into corresponding spacing holes of the first positioning slot.

According to one embodiment of the present invention, said second positioning slot further includes a central slot and at least one spacing hole, and said second positioning component further includes at least one positioning convex which can be inserted respectively into corresponding spacing holes of the second positioning slot.

According to one embodiment of the present invention, the interior surfaces of said first and second positioning slot all configure screw threads, and the exterior surfaces of one end portion of the first positioning slot which can be combined with said first positioning component configure screw threads and the exterior surfaces of said first end portion of the second positioning component configure screw threads.

### BRIEF DESCRIPTION OF THE DRAWINGS

FIG. 1 illustrates the bone fracture surgical method of U.S. Pat. No. 5,951,557;

FIG. 2 illustrates the appearance of bone plate positioning device according to the present invention;

FIG. 3 illustrates the appearance of the first positioning component according to the present invention;

FIG. 4 illustrates the method of operating the bone plate positioning device according to the present invention;

FIG. 5 illustrates the method of operating the bone plate positioning device according to the present invention;

FIG. 6 illustrates the method of operating the bone plate positioning device according to the, present invention;

FIG. 7 illustrates the method of operating the bone plate positioning device according to the present invention;

FIG. 8 illustrates the method of operating the bone plate positioning device according to the present invention;

FIG. 9 illustrates the method of operating the bone plate positioning device according to the present invention;

FIG. 10 illustrates the method of operating the bone plate positioning device according to the present invention;

FIG. 11 illustrates the appearance of the first positioning component according to another embodiment of the present invention; and

FIG. 12 illustrates the appearance of the bone plate according to another embodiment of the present invention.

### DETAILED DESCRIPTION OF THE INVENTION

The present invention relates to an assisted positioning device for bone plate comprising a bone plate and two positioning component sets. To operate it with X-rays, the design of the bone plate will let the bone plate be positioned with bone accurately without larger incisions as before.

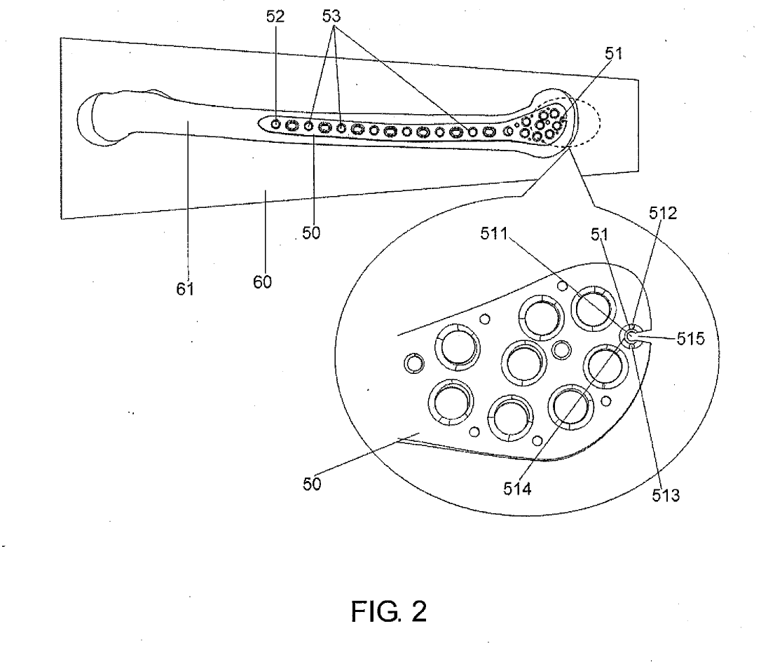

As shown in FIG. 2, an appearance of a bone plate 50 of the assisted positioning device, the bone plate 50 having a several positioning grooves 53, and one end of the bone plate configures a first positioning slot 51 and the other end portion of the bone plate 50 configures a second positioning slot 52. The first positioning slot 51 includes a gap 515 which can link to others. The interior surfaces of the first positioning slot 51 and the second positioning slot 52 configure screw threads that can allow the first positioning slot 51 and the second positioning slot 52 to be combined with two positioning components respectively. In addition, the first positioning slot 51 comprises a central slot 511, at least one spacing hole and a spacing convex rib 514 around the central slot 511 in order to correspondingly connect the positioning components of different standards. In this embodiment of the present invention, these spacing holes will be illustrated by a first spacing hole 512 and a second spacing hole 513 for instance, but the present invention does not limit the number of spacing holes.

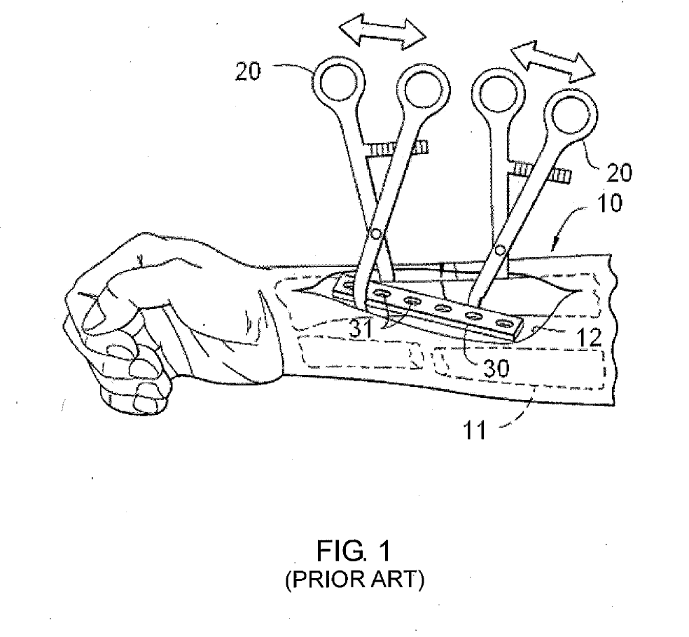

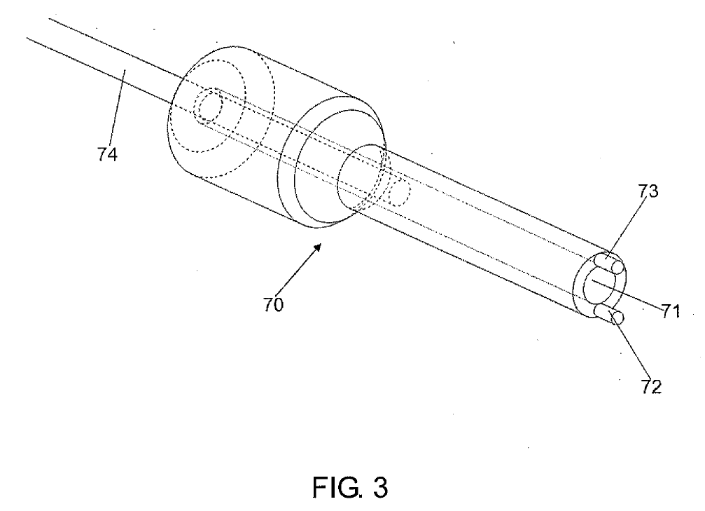

FIG. 3 illustrates the appearance of the first positioning component according to the present invention. Referring to FIG. 1 at the same time, the first positioning component 70 is having a first through slot 71 configured in central axis of the first positioning component 70, and the first through slot 71 can allow a first positioning pin 74 to insert inside. This first positioning pin 74 refers to Kirschner pin which is usually seen in normal bone fracture surgery. The first positioning pin 74 can penetrate the first through slot 71 and the first positioning slot 51 when one end portion of the first positioning component 70 is combined with the first positioning slot 51. Additionally, the first positioning pin 74 can be inserted into corresponding gap 515 for stabilizing and positioning since the size of the first positioning pin 74 is smaller than gap 515. The interior surface of the first positioning slot 51 configures screw threads, and the exterior surface of one end portion of the first positioning component 70 also configures screw threads, and they can match each other. When the first positioning slot 51 comprises a central slot 511, a first spacing hole 512, a second spacing hole 513, and a spacing convex rib 514 which is placed around the central slot 511, the first positioning component 70 has a central slot 511 which can allow the first positioning pin 74 to insert inside and the end portion of the first positioning component 70 is combined with the first positioning slot 51 includes at least one positioning convex. At this embodiment, we will illustrate this positioning convexes by a first positioning convex 72 and a second positioning convex 73 for instance, but we will not limit it. The first positioning convex 72 and the second positioning convex 73 can be inserted into the first spacing hole 512 and second spacing hole 513 of the first positioning slot 51 respectively, and it makes the first positioning component 70 and the first positioning slot 51 be combined with together stably without moving. When the first positioning component 70 is hit by external force accidentally, if we only rely on the first positioning convex 72 and the second positioning convex 73 to position it, it will let them be broken or damaged easily because of the impact strength is all gathering on the contact surface which consists of the first positioning convex 72, the second positioning convex 73, the first spacing hole 512 and the second spacing hole 513. Therefore, the spacing convex rib 514 which is placed around the central slot 511 can separate the impact strength into the interior surfaces of the first 72 and second 73 positioning convexes, or even separate the impact strength into the interior surface of the central slot 511 of the first positioning component 70. It can avoid gathering impact strength on only one spot, and lengthen the first positioning component 70 service lifetimes. In this embodiment of present invention, it will be illustrated by two spacing holes and two corresponding positioning convexes for instance, and we can also use three spacing holes and three corresponding positioning convexes or four spacing holes and four corresponding positioning convexes. We will not limit the quantities of spacing holes and corresponding positioning convexes.

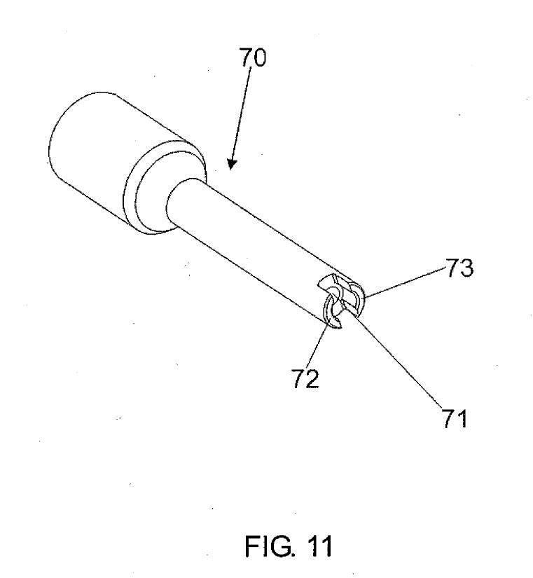

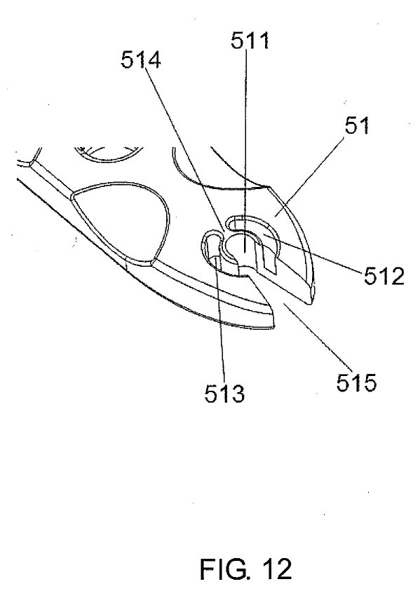

In another embodiment of present invention, the collocation of the first positioning component and the first positioning slot could be another form, and please refer to FIGS. 11 and 12. The first positioning slot 51 comprises a central slot 511, a spacing convex rib 514 which is placed around central slot 511 and a gap 515 that can link outwards. The spacing convex rid 514 makes the first positioning slot 51 become two half-mooned shaped spacing holes—the first 512 and the second 513 spacing holes; said first positioning component 70 configures with a first through slot 71 in central site and two half-mooned shaped first 72 and second 73 positioning convexes. The first 72 and second 73 positioning convexes can be coupled respectively with the first 512 and second 513 spacing holes on the first positioning slot 51 for making the combination of the first positioning component 70 and first positioning slot 51 stable without moving. The spacing convex rib 514 also can make the first positioning component 70 and the first positioning slot 51 more stable without whirling. In this embodiment of present invention, we illustrate it by a spacing convex rib which becomes two spacing holes corresponding with two positioning convexes for instance, and we can let it be three or four. We will not limit the quantities of the spacing convex ribs, spacing holes and positioning convexes.

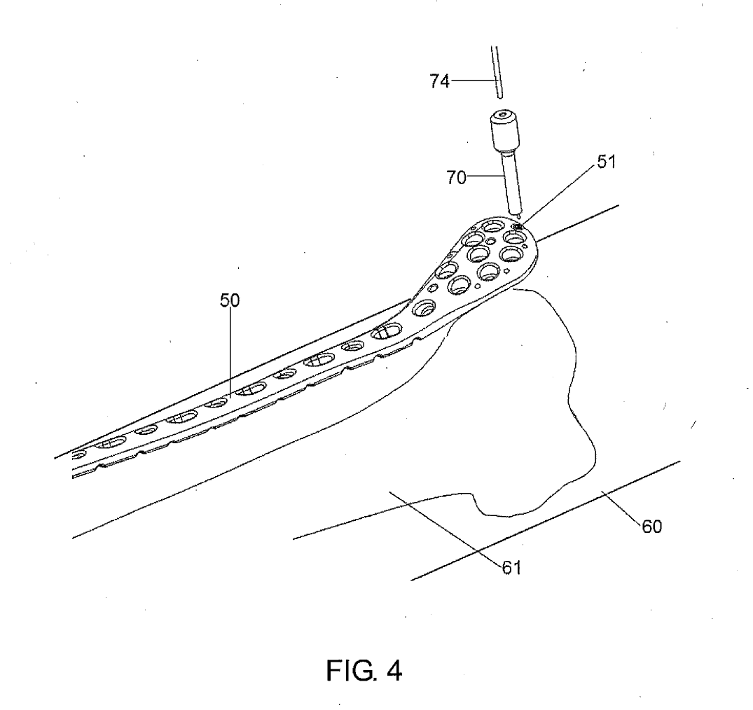

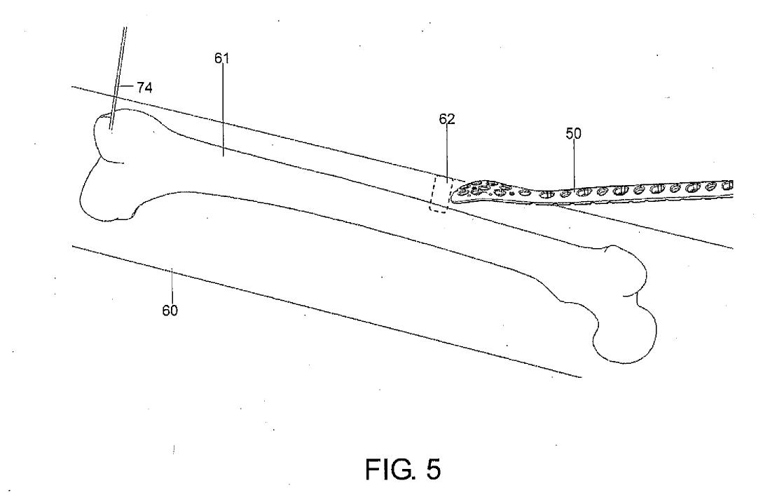

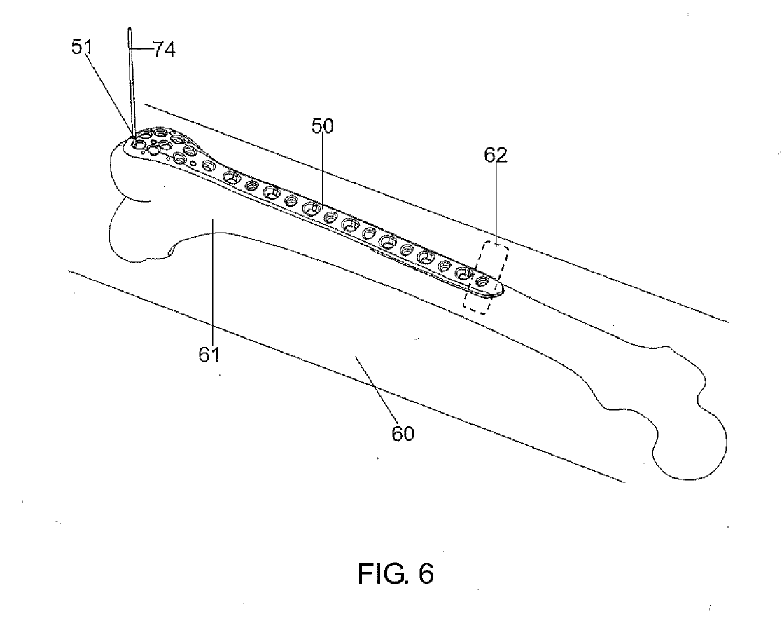

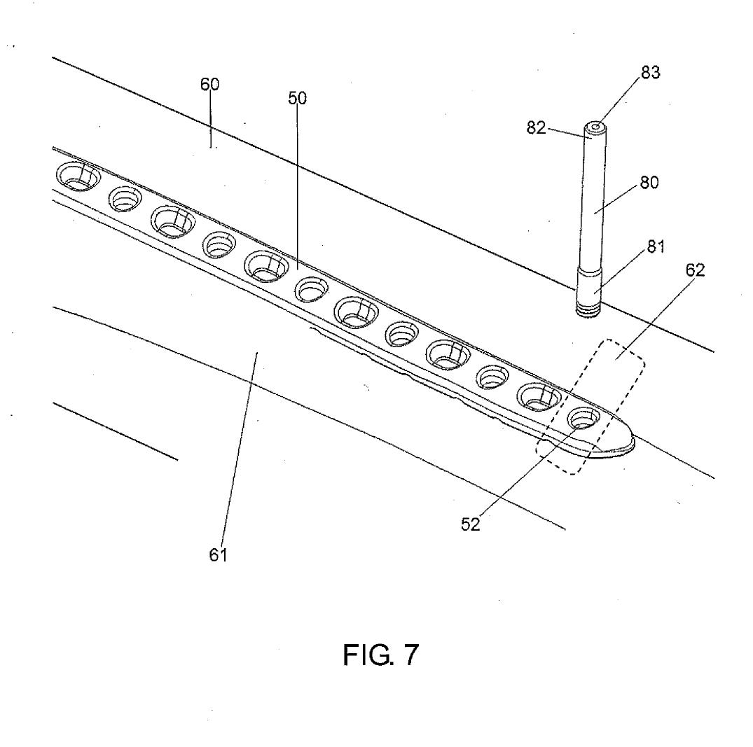

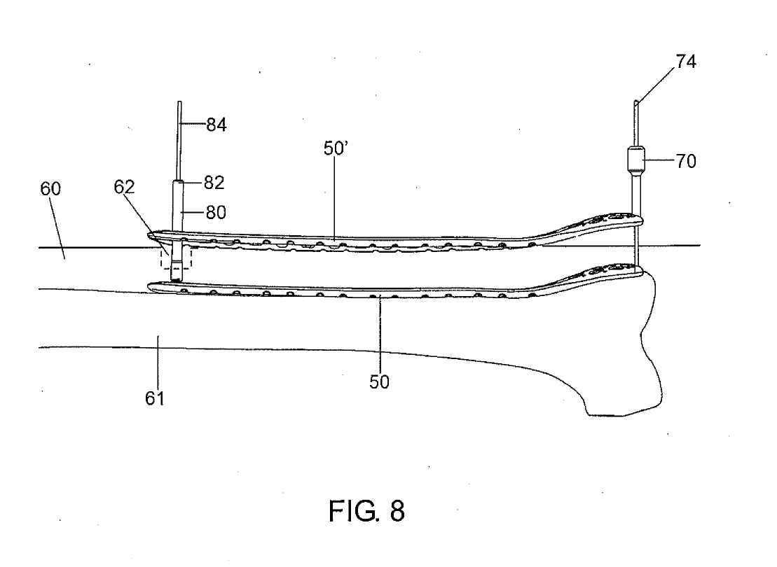

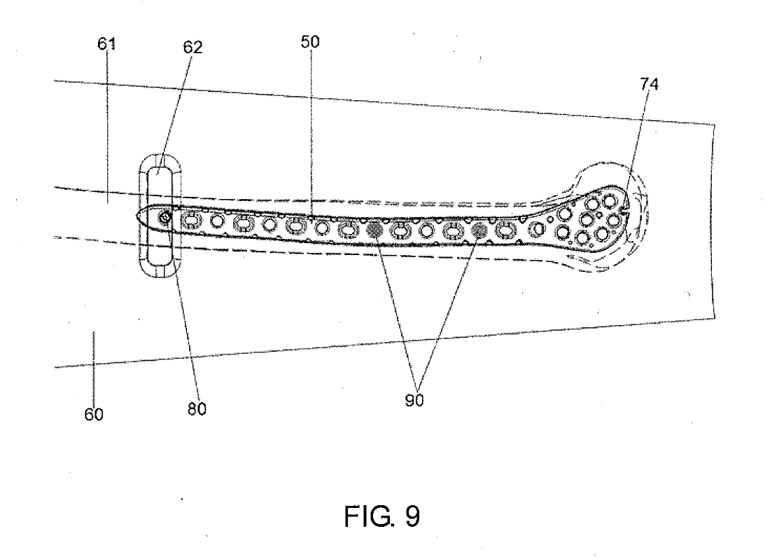

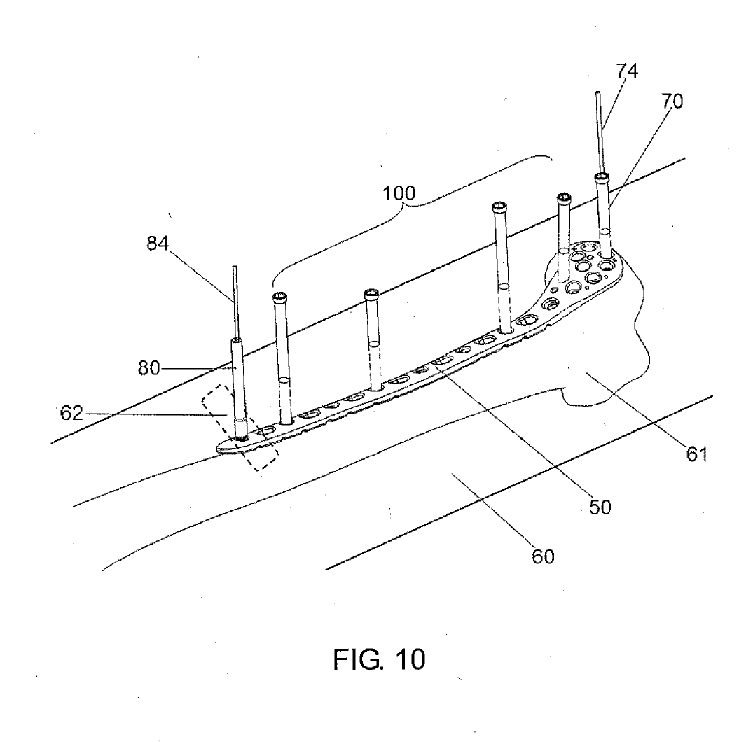

FIGS. 4 to 10 illustrate the operating method according to present invention. As shown in drawings, refers to FIGS. 2 and 3 at the same time, we can place a suitable size bone plate 50 on the fractured bone at first and confirm the relative position of bone plate 50 and bone 61 by X-rays when a bone 61 fractures in a patient's arm 60 (It is not limited to arms which also could be legs). Because of the function of fluoroscopy, X-rays can show exactly where the fracture is and the best combination position of bone plate 50 and bone 61. After confirming this position, it can allow one end portion of the first positioning component 70 to be combined with the first positioning slot 51 to become one integration (by bolt-on or the combination type of first 72 and second 73 positioning convexes) and the first positioning pin (Kirschner pin) 74 can penetrate the first through slot 71 of the first positioning component 70 and the first positioning slot 51, and then we insert the first positioning pin 74 into bone 61 by rotating. It might cause a little vibration when the first positioning pin (Kirschner pin) 74 is inserted into bone 61 by rotating, this vibration might cause distortion, or destroy the penetration point, or destroy the interior surface of the first positioning slot 51. Because the combination of the first positioning component 70 and the first positioning slot 51 is combined stably and the first through slot 71 limits the shaking space of the first positioning pin (Kirschner pin) 74, so it can avoid the vibration when inserting the first positioning pin 74 into bone 61.

The relative position of the first positioning pin 74 and bone 61 are confirmed when we insert the first positioning pin 74 into the bone 61. Then we separate the first positioning component 70 from the first positioning slot 51. We can move the bone plate 50 when the first positioning pin 74 is moved out of the first through slot 71. As the result of the size of gap 515 of the first positioning slot 51 on bone plate 50 is designed to be bigger than the first positioning pin 74, when we move the bone plate 50, the first positioning pin 74, whose position is already confirmed can pass through the gap 515 and the first positioning pin 74 is separated from the bone plate 50. After the first positioning pin 74 is separated from the bone plate 50, paramedics can cut a incision 62 on patient's arm 60 and insert the bone plate 50 into the arm 60 through this incision 62. Because of the first positioning pin 74 being inserted into the bone 61, when we insert the bone plate 50 into the arm 60, we should move the bone plate 50 until the first positioning pin 74 pass through the gap 515 and the first positioning pin 74 is inserted into the first positioning slot 51 again. Then we use the x-rays to confirm whether the position of the bone plate 50 and the first positioning pin 74 are correct or not. After that we can announce one end portion of the bone plate 50 and bone 61 are accurately positioned preliminary.

After we finish positioning one end portion of the bone plate 50 and bone 61, it will need to position the other end portion of the bone plate 50 and bone 61. The relative position of bone plate 50 and bone 61 won't be changed when two end portions of the bone plate are all positioned completely. We need to notice that the distance between first positioning pin 74 and central point of incision 62 is the same as the length of bone plate 50. Then we will make it more convenient to position one end portion of bone plate 50 and bone 61. After confirming the positioning spot, a second positioning component 80 will be combined with a second positioning slot 52 as a integration. A second positioning component 80 comprises a first end portion 81, a second end portion 82 and a second through slot 83 in central site for connecting the first 81 and second 82 end portions, which allows a second positioning pin (Kirschner pin) 84 inside the second through slot 83 to move. And when the first end portion 81 is combined with the second positioning slot 52, it can let the second positioning pin 84 penetrate the second through slot 83 and the second positioning slot 52. The size of second end portion 82 of the second positioning component 80 is smaller than the second positioning slot 52. Because of the exterior surface of first end portion 81 of second positioning component 80 and the interior surface of second positioning slot 52 all configure screw threads, when them are combined as a integration, the structure is stable that it won't loose easily when hit by external force.(The second positioning component 80 and the second positioning slot 52 can each have the same structure as said the first positioning component 70 and the first positioning slot 51. As the combination structure type of the first spacing hole 512 and the first positioning convex 72, according to present invention there is no limit whether the second positioning component 80 needs the same structure or not.) When the second positioning component 80 is combined with the second positioning slot 52 as a integration, we can let the second positioning pin (Kirschner pin) 84 pass through the second through slot 83 of the second positioning component 80 and the second positioning slot 52, and then the second positioning pin 84 is inserted into the bone 61 by rotating to finish positioning the bone plate 50 and the two sides of bone 61.

When we finish positioning two sides of bone plate 50 and bone 61, we can do next step of surgery by using another bone plate 50′(The size and appearance is totally the same as bone plate 50). Because of the size of second end portion 82 of the second positioning component 80 is smaller than the second positioning slot 52, we can easily insert the second end portion 82 of the second positioning component 80 into the second positioning slot(not shown in Fig.) of the bone plate 50′. And the first positioning slot(not shown in Fig.) of the bone plate 50′ also can be inserted into the first positioning pin 74 through its gap(not shown in Fig.), and then we can combine the first positioning component 70 with the first positioning slot(not shown in Fig.) of the bone plate 50′ for avoiding the movement of bone plate 50′. At this time, the bone plate 50 is combined with bone 61 inside patient's arm 60, and the bone plate 50′ is combined with patient's arm 60, and the bone plate 50′ is on the top of patient's arm 60. Viewing from top to down, bone plate 50 and 50′ will be a superposition from up and down, and both the first positioning slot, the second positioning slot and each positioning slots will be overlapped to each other.

After the first and second positioning slots, and each positioning grooves of bone plate 50 and 50′ are overlapping, we can decide which positioning grooves(not shown in Fig.) need to be used according to the position of each positioning grooves of bone plate 50′. When the positioning grooves(not shown in Fig.) which we want to use is confirmed, we can mark some symbols 90 in patient's arm 60 skin corresponding to the positioning grooves(not shown in Fig.) which is used on bone plate 50′, and then we separate one end portion of bone plate 50′ from the first positioning component 70 and the first positioning pin 74, and separate the other end portion from the second positioning component 80. At this time, there are some symbols 90 left on patient's arm 60 skin. Paramedics can cut the patient's arm 60 skin according to those symbols 90, making a small incision until showing the corresponding positioning grooves 53 of bone plate 50. After that, paramedics can combine some bone screws 100 with each corresponding positioning grooves 53 through those small incisions which are corresponding to each symbols 90, and the bone screws pass through each positioning grooves 53 and are inserted into bone 61. It can combine the bone plate 50 with bone 61 strongly by the bone screws 100 to assemble the fractured bone 61. The interior surfaces of each positioning grooves 53 configure screw threads for increasing the stability of bone screws 100 whose exterior surface configure screw threads. After each bone screws 100 are combined with the corresponding positioning grooves 53 as a integration, paramedics can separate the second positioning component 80 and the first positioning pin 74 from bone plate 50 or bone 61 through the second positioning slot 52 and the first positioning slot 51 respectively, and then stitch the incision 62 and those small incisions to finish the fractured bone surgery.

Using this assisted positioning device for bone plate of present invention in fractured bone surgery can reduce the size of the patient's incisions, and avoid damaging the soft tissue and muscle and shorten healing time after surgery. Because the size of incisions becomes small, the bone fracture surgery will be more acceptable by the patients, and it has two advantages, more beautiful and less painful. Furthermore, the whole bone fracture surgery time will be reduced because the simplification of an assisted positioning device for bone plate of present invention which makes the paramedics do surgery more easily and decrease the surgical expenses, and it can let the medical sources to be use in more important places.

While the foregoing description discloses the preferred embodiments of the present invention in particular detail, it must be understood that numerous modifications, substitutions and changes can be undertaken without departing from the true spirit and scope of the present invention as defined by the ensuing claims. The invention is therefore not limited to specific preferred embodiments as described, but is only limited as defined by the following claims.

What claimed is:

1. An assisted positioning device for bone plate, said device comprising:
a bone plate, having several positioning grooves in the surface in order to correspond with a number of bone screws provided for the integration, and one end portion of said bone plate configures a first positioning slot, and the other end portion of said bone plate configures a second positioning slot, and said first positioning slot includes one gap;
a first positioning component, having a first through slot configured in central axis of the first positioning component, and said first through slot can allow a first positioning pin to insert inside; and
a second positioning component, having a first end portion and a second end portion, and said second positioning component configures a second through slot in central axis of the second positioning component to connect said first end portion and said second end portion, and said second through slot can allow a second positioning pin to insert inside

2. The assisted positioning device for bone plate according to claim 1, wherein:
when one end portion of said first positioning component is combined with said first positioning slot, said first positioning pin can penetrate said first through slot and said first positioning slot, and said first positioning pin size is smaller than said gap.

3. The assisted positioning device for bone plate according to claim 1, wherein:
when said first end portion of the second positioning component is combined with said second positioning slot, the second positioning pin can penetrate said second through slot and said second positioning slot, and said second end portion size is smaller than said second positioning slot.

4. The assisted positioning device for bone plate according to claim 1, wherein:
said first positioning slot further includes a central slot and at least one spacing hole, and said first positioning component further includes at least one positioning convex which can be inserted respectively into corresponding spacing holes of said first positioning slot.

5. The assisted positioning device for bone plate according to claim 4, wherein:
said first positioning slot further includes a spacing convex rib which is placed around said central slot.

6. The assisted positioning device for bone plate according to claim 1, wherein:
the interior surface of said first positioning slot configures screw threads, and the exterior surface of the combination of said first positioning component and said first positioning slot also configures screw threads.

7. The assisted positioning device for bone plate according to claim 1, wherein:
the interior surface of each said positioning grooves and the exterior surface of each bone screws all configure screw threads.

8. The assisted positioning device for bone plate according to claim 1, wherein:
said second positioning slot further includes a central slot and at least one spacing hole, and said second positioning component further includes at least one positioning convex which can be inserted respectively into corresponding spacing holes of said second positioning slot.

9. The assisted positioning device for bone plate according to claim 8, wherein:
said second positioning slot further includes a spacing convex rib which is placed around said central slot.

10. The assisted positioning device for bone plate according to claim 1, wherein:
the interior surface of said second positioning slot configures screw threads, and the exterior surface of said first end portion of said second positioning component also configures screw threads.
